(19) United States
(12) Patent Application Publication
(10) Pub. No.: US 2018/0357384 A1
(43) Pub. Date: Dec. 13, 2018

(54) UNIVERSAL INTERFACE FOR DENTAL DEVICES

(71) Applicant: KAVO DENTAL TECHNOLOGIES, LLC, Charlotte, NC (US)

(72) Inventors: Robert Thomas St. Louis, Charlotte, NC (US); Michael Carl Dunaway, Charlotte, NC (US); Steven Bohon, Charlotte, NC (US)

(21) Appl. No.: 15/618,045

(22) Filed: Jun. 8, 2017

(51) Int. Cl. G06F 19/00; A61B 6/14; A61B 6/00; A61C 17/02; A61C 1/00; A61B 1/24; A61C 1/08; A61G 15/02; A61C 5/50; A61C 19/00

(52) U.S. Cl. CPC G06F 19/3406; A61B 6/14; A61B 6/54; A61C 17/02; A61C 19/002; A61B 1/24; A61C 1/088; A61G 15/02; A61C 5/50; A61C 1/0015

(57) ABSTRACT

A universal controller for controlling dental devices includes a memory having a graphical user interface generator and control software for a plurality of dental devices. The universal controller includes a transceiver, a display for displaying graphical user interfaces and for receiving inputs, and an electronic processor connected to the memory, the transceiver, and the display. The electronic processor is configured to provide a graphical user interface to the display in response to a selection received from the display. The universal controller is configured to display a plurality of dental devices to be controlled and enable selection thereof. Further, multiple devices are displayable on the universal controller, along with a patient information.

### BACKGROUND

Embodiments relate to a single universal interface, such as a portable universal controller, that is configured to control a plurality of dental devices including a delivery unit with a handpiece.

### SUMMARY

In one embodiment, a universal controller for controlling dental devices comprises a memory that includes a graphical user interface generator and control software for a plurality of dental devices, a transceiver, a display for displaying graphical user interfaces and for receiving inputs, and an electronic processor connected to the memory, the transceiver, and the display. The electronic processor is configured to provide a graphical user interface to the display in response to a selection received from the display.

In another embodiment, a system for controlling a plurality of dental devices comprises a universal controller and a dental device. The universal controller includes a memory that includes a graphical user interface generator and control software for the plurality of dental devices, a transceiver, a display for displaying graphical user interfaces and for receiving inputs, and an electronic processor connected to the memory, the transceiver, and the display. The dental device includes an electronic controller for controlling the dental device, a transceiver for wireless communication with the universal controller, and at least one device actuator. The electronic controller is configured to control the device actuator to operate the dental device in response to an input from the universal controller.

In one embodiment, a method for controlling dental devices with a universal controller includes determining dental devices located in an area for communication therewith, displaying a graphical user interface on the universal controller of a plurality of dental devices in the area that are capable of being controlled, and in response to selection of one of the dental devices, displaying a graphical user interface on the universal controller that includes control inputs for the selected dental device. The method further includes, in response to selection of one of the control inputs on the graphical user interface, transmitting a control signal to the selected dental device for operating the selected dental device.

Other aspects will become apparent by consideration of the detailed description and accompanying drawings.

### BRIEF DESCRIPTION OF THE DRAWINGS

FIG. 1 illustrates a perspective view of a treatment unit and a portable universal controller.

FIG. 2 is a block diagram of the universal controller.

FIG. 3 is a block diagram of a delivery unit.

FIG. 4 is a perspective view of a delivery unit having the universal controller, but no display.

FIG. 5 illustrates a CBCT machine and the universal controller.

FIG. 6 illustrates a docking station and the universal controller.

FIG. 7 is a communication diagram for components including the universal controller, a dental handpiece maintenance system, a dental handpiece system, a delivery unit, logging equipment, a desktop computer, and a cloud server.

FIG. 8 illustrates a flow chart for operation of the portable universal controller.

FIG. 9 illustrates a graphical user interface for display by the portable universal controller.

FIG. 10 illustrates a graphical user interface for display by the universal controller.

FIG. 11 illustrates a second flow chart for a second operating embodiment for the universal controller.

FIG. 12 illustrates a graphical user interface for display by the universal controller during an Endodontics procedure.

FIG. 13 illustrates a graphical user interface for display by the universal controller that includes display of an X-ray image.

FIG. 14 illustrates a graphical user interface for display by the universal controller that includes display of an irrigation display window.

### DETAILED DESCRIPTION

Before any embodiments are explained in detail, it is to be understood that they are not limited in their application to the details of construction and the arrangement of components set forth in the following description or illustrated in the following drawings. Other embodiments are possible and embodiments explained are capable of being practiced or of being carried out in various ways.

Some embodiments described herein may be implemented as a non-transitory, computer-readable medium storing instructions executable by one or more electronic processors to perform the described functionality. As used in the present application, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

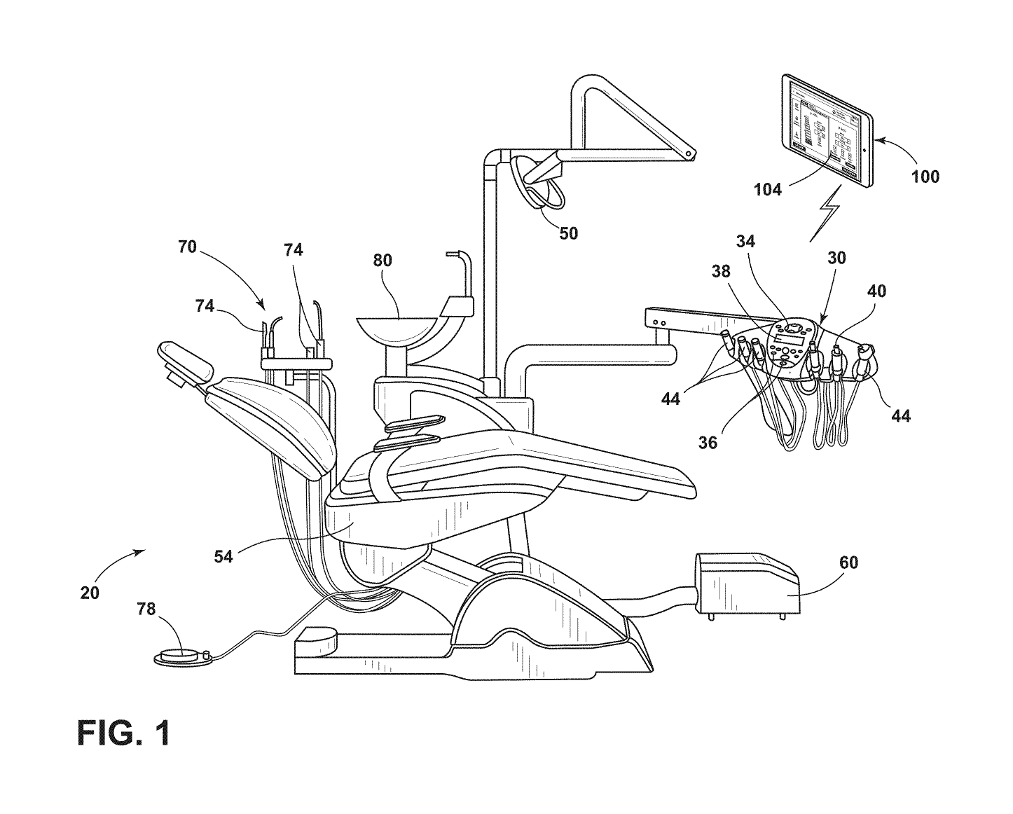

FIG. 1 illustrates an embodiment of a treatment unit 20 that includes a delivery unit 30 having an electronic controller 34 with input controls 36 and a display 38. The delivery unit 30 includes a tray 40 that receives a plurality of instruments 44. The treatment unit 20 includes a light 50, a powered treatment chair 54 and a junction box 60. The junction box 60 provides connections for at least one of a group consisting of: a water or fluid source, a compressed air source, and a vacuum source Further, the treatment unit 20 includes a second delivery unit 70 with additional instruments 74 and a footswitch 78. A spittoon 80 is provided in a dental treatment room with the treatment unit 20.

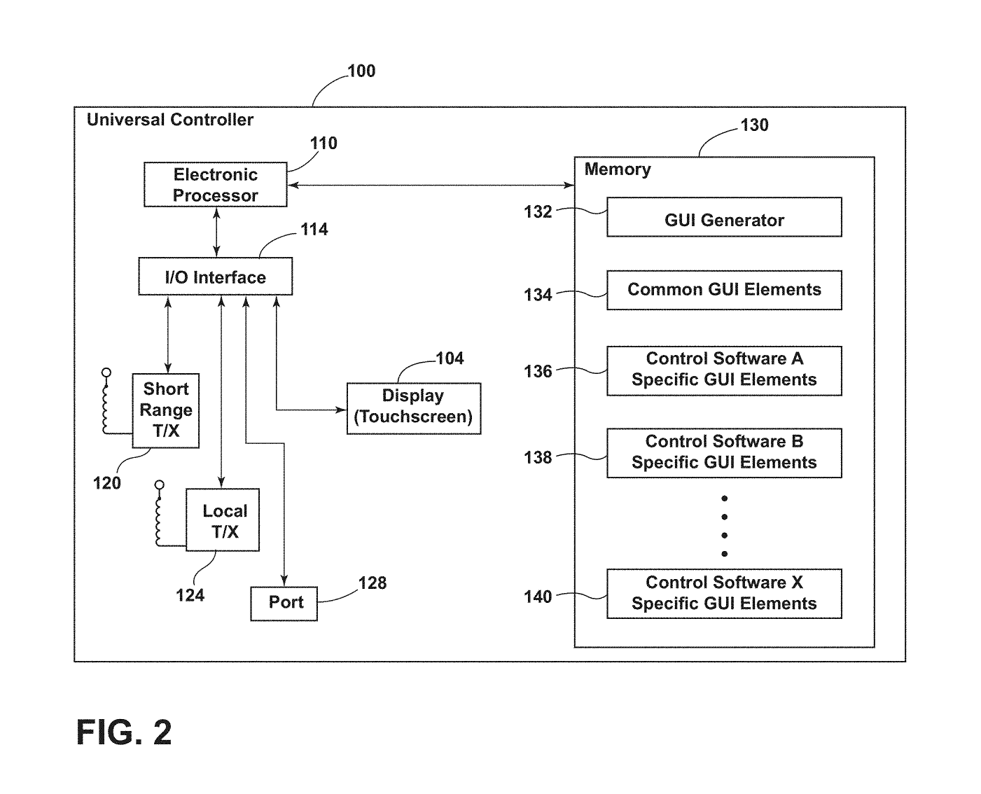

FIG. 1 also illustrates a portable universal controller 100. In the example, illustrated the portable universal controller 100 includes a display 104 that also acts as an input device. In one embodiment, the display 104 is a touchscreen that produces electrical outputs in response to touches by a user. The portable universal controller 100 illustrated in FIG. 2 also includes an electronic processor 110 that is connected to the display 104 via an input/output interface 114. Switches, such as an on/off button (not shown), may be provided on a housing of the universal controller 100. The universal controller 100 also includes a short range transceiver 120 for communicating with the delivery units 30, 70 or other devices to be controlled. The short range transceiver 120 may include a one or more of a Bluetooth transceiver, a near-field communications (NFC) transceiver, and another suitable wireless transceiver.

The universal controller 100 also includes a local transceiver 124 to communicate with the delivery units 30, 70 or with other devices. The local transceiver 124 communicates as one of a WI-FI 802.11 transceiver, a radio frequency (RF) transceiver, or another transceiver. The universal controller 100 also includes at least one port 128, such as an electrical socket, for receiving information or outputting information or other data via a hard wire connection with a communication connector or cable.

FIG. 2 also shows a memory 130 or other computer-readable medium in communication with the electronic processor 110. In some embodiments, the input/output interface 114 includes a communication bus connected to various devices that utilize commands from the electronic processor 110. The memory 130 includes a graphical user interface (GUI) generator 132 for generating graphical user interface elements 134 for selective use on the display 104. The memory 130 stores control software (A) 136, control software (B) 138, and control software (X) 140 as software to be selectively executed depending on the dental device A, B, . . . , X selected for control by the universal controller 100.

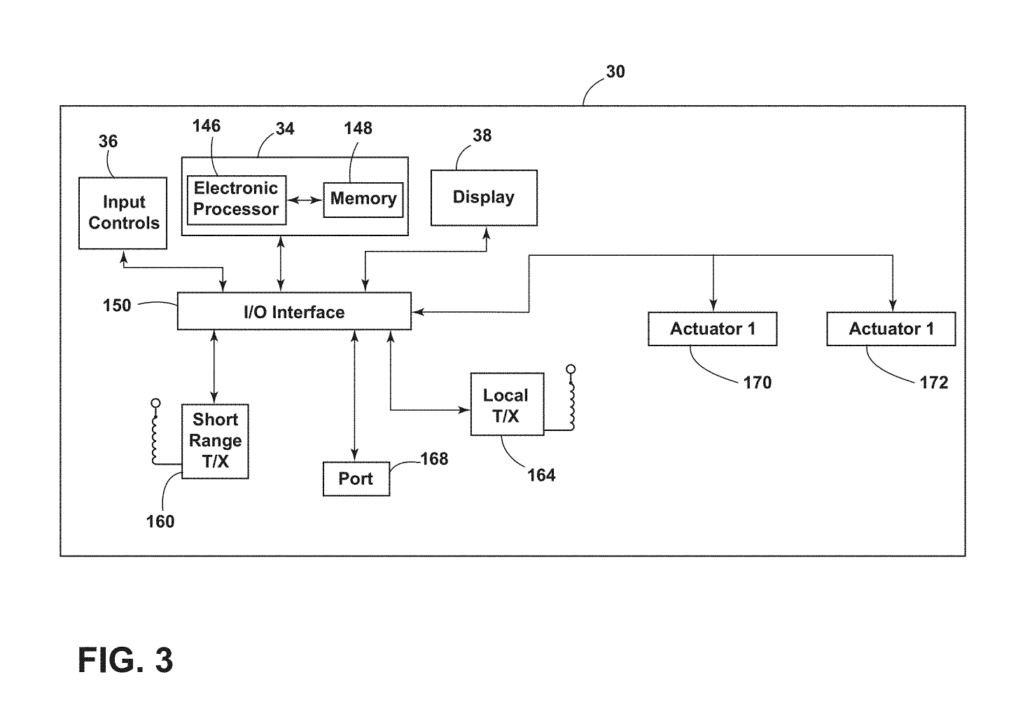

FIG. 3 is a block diagram of electrical components of the delivery unit 30 illustrated in FIG. 1. In one embodiment, the delivery unit 30 includes an electronic controller 34 that has an electronic processor 146 and a memory 148. An input/output interface 150 may include a communication bus that provides communication between the electronic processor 146 and the input controls 36, the display 38, and the memory 130.

The delivery unit 30 illustrated in FIG. 3 includes a short range transceiver 160 for wireless communication with the universal controller 100. The short range transceiver 160 may include one or more of a Bluetooth transceiver, a near-field communications (NFC) transceiver, and another suitable wireless transceiver for direct communication with the short range transceiver 120 of the universal controller 100.

The delivery unit 30 illustrated in FIG. 3 also includes a local transceiver 164 to communicate with the universal controller 100 or other devices. The local transceiver 164 may be configured to communicate with a WI-FI 802.11 network access point, a radio frequency (RF) transceiver, or another communication network. The delivery unit 30 also includes a port 168 for receiving information or outputting information or other data via a hard wire connection with a communication connector or cable.

The delivery unit 30 shown in FIG. 3 is designed to control device actuators (for example, to control motors in or associated with a dental instrument. The embodiment illustrated includes a first device actuator 170 that controls rotational speed and torque of a motor in response to commands from the electronic processor 146. A second device actuator 172 controls irrigation or suction for selected instruments 44 shown in FIG. 1. Other embodiments including additional device actuators for additional instruments, such as handpieces of the delivery unit 30, are contemplated. Thus, other embodiments are directed to at least one device actuator, or more device actuators.

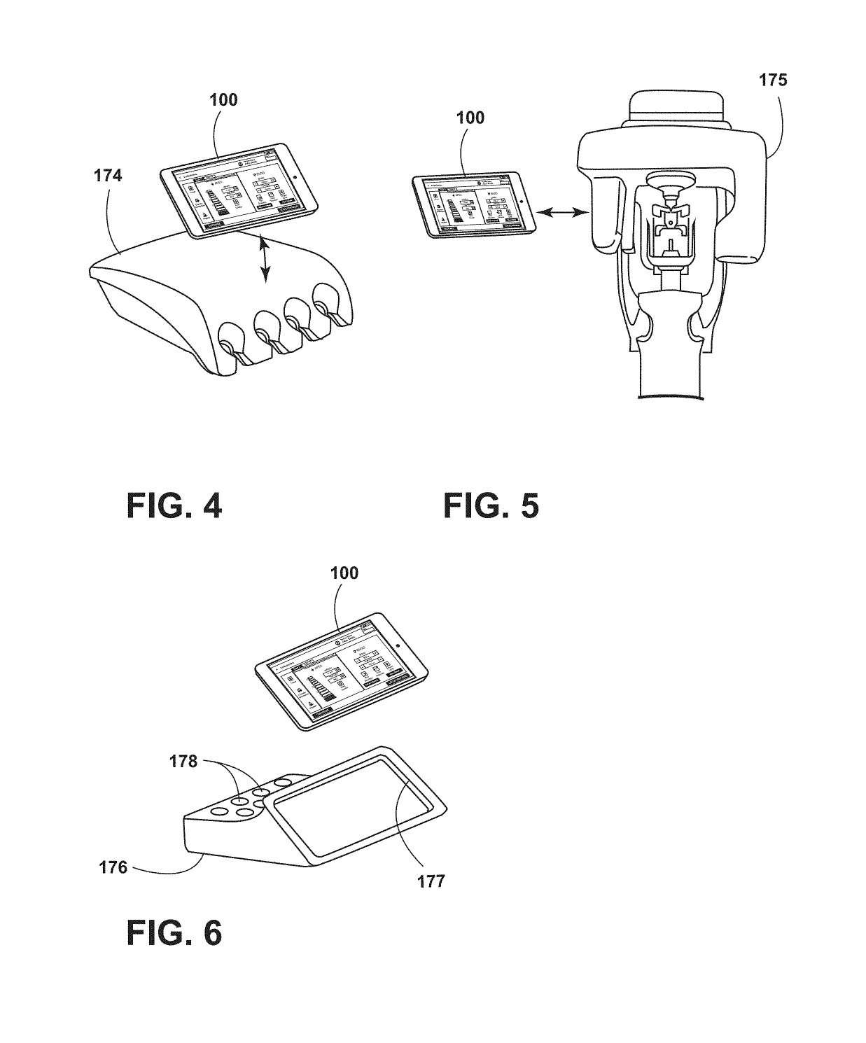

FIG. 4 shows a delivery unit 174 that is free from or otherwise lacks a display. The delivery unit 174 also lacks input mechanisms for providing information to an electronic processor or similar device. Instead, the delivery unit 174 includes components corresponding to the electronic processor 146, the memory 148, the input/output interface 150, the short range transceiver 160, the local transceiver 164 and the port 168 shown in FIG. 3. In this embodiment, there is no display, as the display 104 of the universal controller 100 acts as a display for the delivery unit 174.

FIG. 5 illustrates a cone beam computed tomography (CBCT) X-ray machine 175, which is an imaging device that is configured to move about a head of a patient to provide a three dimensional image. The universal controller 100 is configured to wirelessly communicate with and display controls for the CBCT X-ray machine 175 in a manner that is similar to how the universal controller 100 communicates with and displays controls for the delivery unit 174.

FIG. 6 shows a docking station 176 that lacks a display and also lacks input mechanisms. The docking station 176 includes a receiving slot 177 for receiving the universal controller 100. In one embodiment, a mechanically-implemented electrical connection between the universal controller 100 and the docking station 176 is provided. In one example, the port 128 of the universal controller 100 has a pin or other element that snaps or locks with an electrical socket of the stand-alone docking station 176 when the universal controller is placed into the receiving slot 177 of the docking station. In another embodiment, the universal controller 100 has one or more electrical sockets and the docking station 176 has one or more pins. The pins are located in the receiving slot 177 so that they mate or lock with the port 128 or ports of the universal controller 100. The docking station 176 includes a number of receptacles 178. The receptacles 178 hold instruments, including handpieces, for charging and storing purposes.

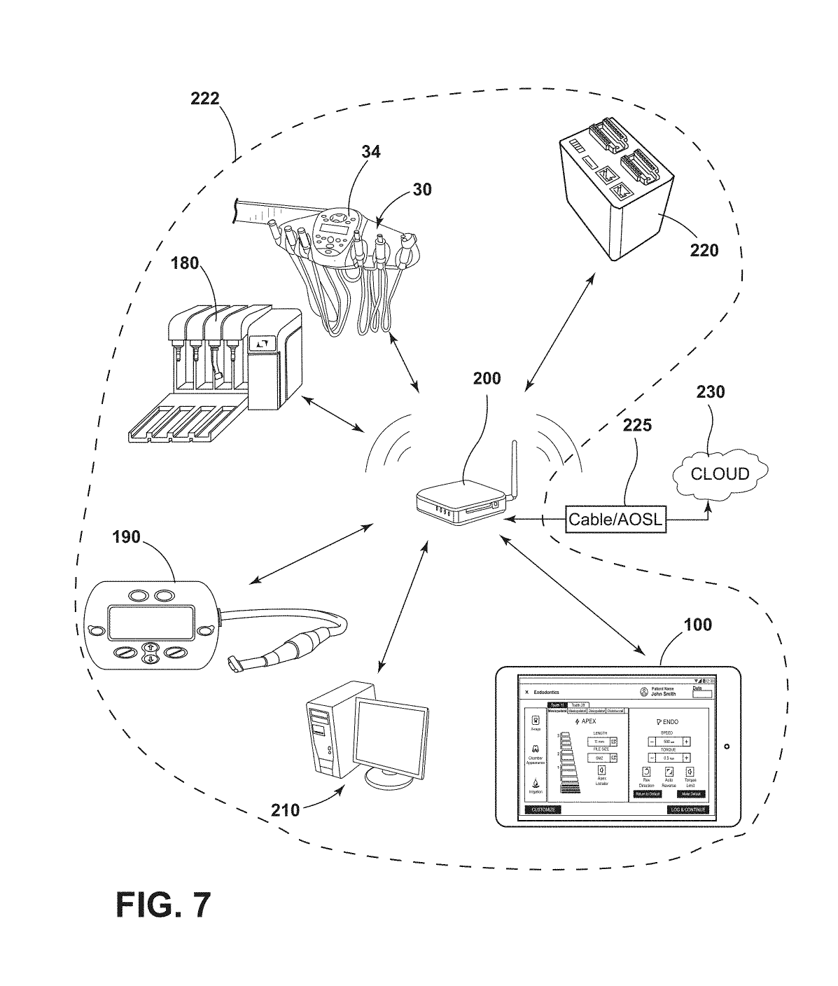

FIG. 7 is a communication diagram illustrating connections and communications paths between the portable universal controller 100 and other devices. FIG. 7 illustrates the portable universal controller 100 with the delivery unit 30, a handpiece maintenance device 180, a dental handpiece system 190, a computer work station 210 and logging equipment 220. The universal controller 100 communicates with these components via a network access point 200 using the local transceiver 124. This group of devices and equipment, in combination with the universal controller 100 and the network access point 200, define a network 222.

In some instances, the universal controller 100 communicates with the delivery unit 30, the handpiece maintenance device 180, the dental handpiece system 190, and in some instances the computer work station 210 and the logging equipment 220, using the short range transceiver 120 and Bluetooth communication. Thus, the universal controller 100 is capable of direct communication with the individual devices without use of the network access point 200. Further, the universal controller 100 may communicate with one or more of the other devices shown in FIG. 7 by an electrical wire connection via the port 128.

The universal controller 100 also communicates via the network access point 200 and via another connection 225, for example, wired internet connection, with a cloud server 230 or cloud computer.

Additional dental devices, such as an X-ray machine, apex locator, light 50, and powered treatment chair 54 of the treatment unit 20, along with additional dental equipment (not shown), are controllable by the universal controller 100 in other embodiments. Thus, the universal controller 100 is capable of communicating with and controlling a plurality of dental devices in an area within a treatment room as further discussed herein.

Operation Of The Universal Controller

Operation of the universal controller 100 requires a user to log in with a username and a password through, for example, one or more dialog boxes in a graphical user interface (GUI) on the display 104. Upon entry of an authorized user name and password, the user obtains access to and control of the universal controller 100. Thereafter, a start-up menu or dash board is provided in the GUI on the display 104 of the universal controller 100.

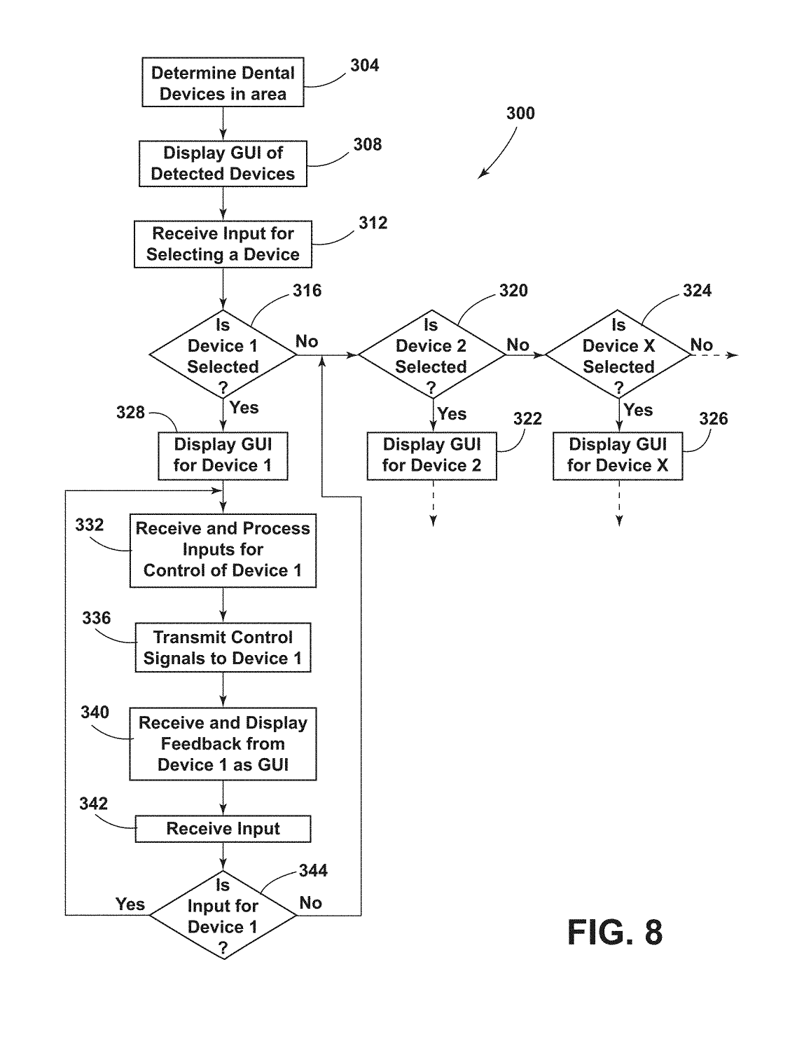

In one embodiment, the user selects, for instance, instrument control. Upon selection, or even before selection upon start-up, the electronic processor 110 of the universal controller 100 executes a method illustrated in the flow chart 300 of FIG. 8. The electronic processor 110 executes the method to determine the dental devices disposed in a treatment area nearby using short range transceiver 120 and to find a connection to a network access point 200 with a local transceiver 124 (step 304). The short range transceiver 120 determines the presence of various devices nearby using a Bluetooth communications or similar communications. The dental devices include a powered treatment chair 54, lights 50, X-ray sensors, 2-D imaging devices, 3-D imaging devices, wireless instruments, delivery units 30, 70, stand-alone dental handpiece systems 190, endodontic obturation systems, electronic apex locators, maintenance devices 180, irrigation devices, and sterilization equipment. In some embodiments, the same or other dental devices are capable of communication with the universal controller 100 via the network access point 200 using WI-FI or a similar communication links. Communications are used to determine the presence of dental devices on the local network 222 via the short range transceiver 120 and/or the local transceiver 124 of the universal controller 100 and the network access point 200 (step 304). Synchronization and communication between the universal controller 100 and all of the devices via the short range transceiver 120 and/or the local transceiver 124 is the result.

The universal controller 100 displays a group of the identified and detected dental devices on the display 104 (step 308 illustrated in FIG. 8). The user selects a dental device from the group of dental devices on the display 104 to provide an input (step 312) to the universal controller 100. In FIG. 8, the selection of dental devices is illustrated as a series of decision boxes in the flowchart for a series of devices, namely device 1 through device x.

The electronic processor 110 of the universal controller 100 determines if the input is for selection of dental device (1) (step 316 in FIG. 8). If not, the electronic processor 110 determines if dental device (2) is selected (step 320). If Yes for dental device (2), the electronic processor displays the GUI for dental device (2) (step 322). If dental device (2) is not selected, the electronic processor 110 determines if dental device (X) is selected (step 324 illustrated in FIG. 8). If Yes for selection of dental device (X), the GUI for device (X) is displayed on the universal controller (step 326). Operating information for each of the devices (device 1 through device x) is stored in the memory 130 of the universal controller 100.

When a particular dental device is selected and identified by the electronic processor 110, control software for the specific selected dental device (device (1) in the example illustrated in FIG. 8) is provided or linked to the electronic processor 110 from the memory 130, and a graphical user interface for the dental device (1) is provided on the display 104 of the universal controller 100 (step 328). Thereafter, the GUI provided on the display 104 is used to receive control inputs that are provided to electronic processor 110 to adjust the control of the dental device (1) and the electronic processor 110 of the universal controller 100 processes the control inputs using the control software for the specific software of the selected dental device (step 332). Then, the electronic processor 110 transmits a control signal to the dental device (1) via one of the short range transceiver 120, the local transceiver 124 and the port 128 (step 336).

The received control signal or control commands operate the dental device, which transmits a return signal as feedback regarding the condition of the dental device that is received and displayed by the universal controller 100 (step 340 illustrated in FIG. 8) as conditions on a graphical user interface. In one embodiment, the method receives an additional input for processing and control of the dental device from the user (step 342). The method determines whether the input is for device (1) (decision step 344). If so, the electronic processor 110 of the universal controller 100 returns to and re-executes step 332, and subsequently steps 336, 340, 342 and again step 344, as illustrated in FIG. 8.

In other instances when a different dental device or other feature has been selected (decision step 344), the method returns to determine if dental device (2) is selected (step 320). Thus, during operation of dental device (1), another device is selected and the electronic processor 110 of the universal controller 100 provides a GUI to the display 104 for the different device from the memory 130. The operations of device (2), following steps 320, 322, are shown in FIG. 8 as broken lines ending in an arrow. The broken lines ending in an arrow represent additional steps (not shown) for device (2) that are similar to steps 332, 336, 340, 342, 344 for the method described for dental device (1). Likewise, following steps 324, 326 shown in FIG. 8, the broken lines ending in an arrow represent additional steps (not shown) for the device (X) that are essentially the same as steps 332, 336, 340, 342, 344 for device (1). Thus, further description of the operation of additional selectable dental devices (2), . . . , (X) is not provided.

In operation, dental device (2), after display of the GUI (step 322), and dental device (X), after display of the GUI (step 326), operate in a similar manner as dental device (1). Thus, further discussion of method steps for dental device (2) and additional dental devices represented by dental device (X) shown in broken line, is not provided.

Multiple Dental Devices—Instrument Control

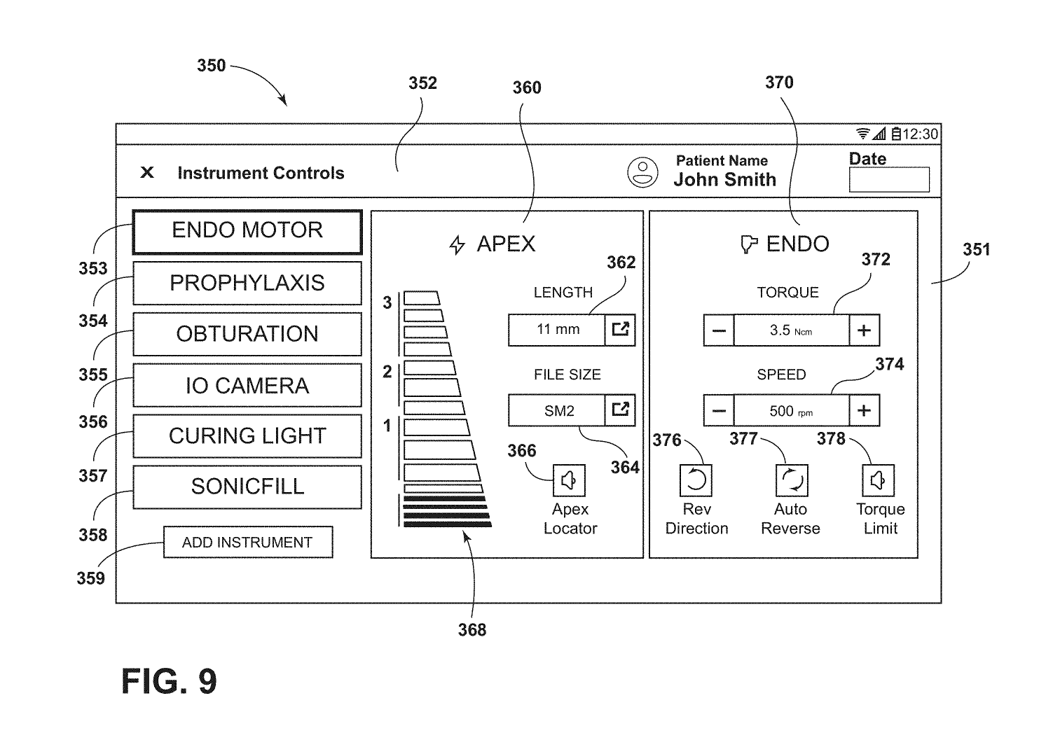

FIG. 9 shows a graphical user interface 350 of instrument controls for the display 104 of the universal controller 100. The GUI 350 of instrument controls includes a browser window 351 and a mode bar 352 that displays the operating mode, the patient name, date, and time. Further, the GUI 350 includes a left column having a vertical list of virtual buttons 353-358 for selection of devices and/or procedures to be controlled. An additional virtual button 359 at the lower left of the GUI 350 is provided to add additional instruments to the list to be controlled. Actuating the add instrument virtual button 359 would result in a new pop-up window or other arrangement provided on the browser window 351 to add an instrument.

A central section of the GUI 350 is directed to a device window 360 for an electronic apex locator. The device window 360 includes a dropdown menu box 362 for generating a list of lengths and for displaying the selected apex length, such as 11 mm. Further, the device window 360 includes a dropdown menu box 364 for generating a list of file sizes and for displaying the selected file size. Further, an apex locator icon 366 is provided in the device window to selectively provide on/off audible alerts for when the apex has been reached. A bar graph 368 displays feedback for apex locator operation.

A right section of the GUI 350 is directed to a device window 370 for control of operating torque and operating speed of the motor for a tool secured to an instrument of a delivery unit 30, 70 or of a handpiece system 190. The device window 370 includes a torque display and selection box 372 having a decrement box (−) and an increment box (+) to obtain inputs for adjusting the maximum torque of the motor. Further, the device window 370 includes a speed display and selection box 374 that displays the speed (rpm) of the motor and includes a decrement box (−) and an increment box (+) for touching to change the speed of the motor. Further, the device window 370 includes a reverse direction button or icon 376 for reversing the direction of the motor and an auto reverse button or icon 377. Finally, the device window 370 includes a torque limit button or icon 378 for selectively providing the on/off alert when the maximum torque has been reached. Thus, a user is capable of selectively controlling two different dental devices, namely the apex locator or the motor in a one-touch fashion as shown in the GUI 350 of FIG. 9.

Accordingly, the GUI 350 illustrated in FIG. 9 shows a split screen mode for the universal controller 100, where one selected dental device is an electronic apex locator as a first dental device having operating conditions displayed in the device window 360 of the GUI 350. One condition displayed by the GUI 350 is a length value for the apex locator and another condition is a file size. The length and file size are recorded and stored in a patient history file in one embodiment. The second dental device is a motor for a handpiece having operating conditions that are displayed in the device window 370 of the GUI 350 shown in FIG. 9 for the universal controller 100. One condition displayed by the GUI 350 for the motor is a maximum operating torque provided in the torque display and selection box 372 and another condition is operating speed provided in the speed display and selection box 374. The GUI 350 provides for selective control for the electronic apex locator and for the motor for an instrument, such as a handpiece of a delivery unit 30, 70 or a dental handpiece system 190.

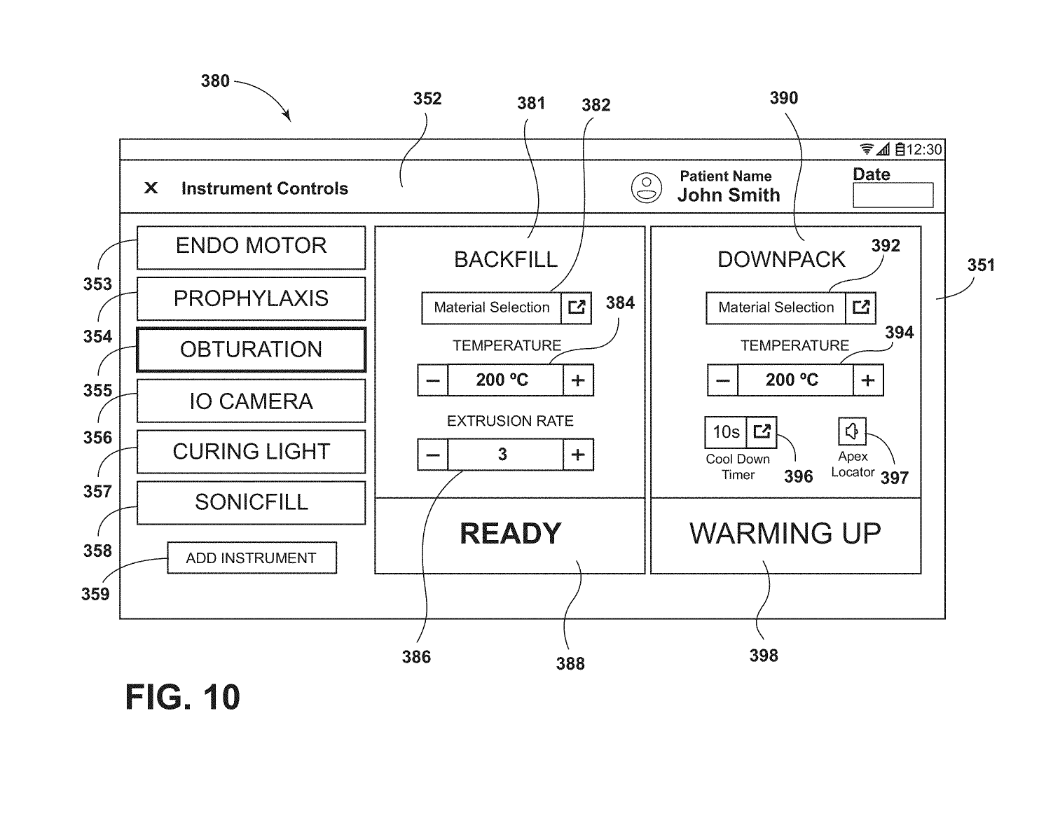

Upon selecting the virtual button 355 from the left column vertical list of the GUI 350 shown in FIG. 9, the universal controller 100 provides the graphical user interface 380 for an obturation system as shown in FIG. 10. The GUI 380 for instrument controls includes the browser window 351 and the mode bar 352 that displays the operating mode, the patient name and date and time. The GUI 380 includes the same vertical list of virtual buttons 353-359 as in FIG. 9, except the virtual button 355 is highlighted.

The central section of the GUI 380 shown in FIG. 10 is directed to a device window 381 of a backfill device for a root canal procedure. The device window 381 includes a dropdown menu box 382 for generating a list of materials and selecting the material used with the backfill device. The device window 381 also includes a temperature display box 384 for displaying a temperature of the material with a decrement box (−) and an increment box (+) for receiving touch inputs to adjust the temperature of the material. Further, the device window 381 includes an extrusion rate display box 386 that includes a decrement box (−) and an increment box (+) for receiving touch inputs to adjust the extrusion rate of material output by the back fill device. Finally, the device window 381 includes a condition bar 388 that displays the condition of the backfill device. Thus, adjustments for the materials, temperature, and extrusion rate of the backfill device are operating conditions that are provided by the graphical user interface 380.

A second device window 390 for a downpack device used in a root canal procedure is provided on the GUI 380 shown in FIG. 10. The device window 390 includes a dropdown menu box 392 for generating a list of materials for selection of a material. The device window 390 includes a temperature display and selection box 394 for displaying the material temperature and a decrement box (−) and an increment box (+) for receiving touch inputs to adjust the temperature of the material. The device window 390 includes a cool down timer dropdown menu box 396 for generating a list of times and selecting a cool down time for the downpack device. The device window 390 also includes an apex locator icon 397 for the apex locator. Finally, the device window 390 includes a condition bar 398 that displays the condition of the downpack device.

In the GUI 380 of FIG. 10, the status of the backfill device “READY” is displayed by the condition bar 388 and the status of the downpack device “WARMING UP” is displayed by the condition bar 398. Again, a selection of devices/procedures is provided in the left column of the GUI 380 illustrated in FIG. 10. Selection of the endo motor virtual button 353 at the top left column in FIG. 10 returns the universal controller 100 to the GUI 350 illustrated in FIG. 9.

Multiple Dental Devices—Procedures

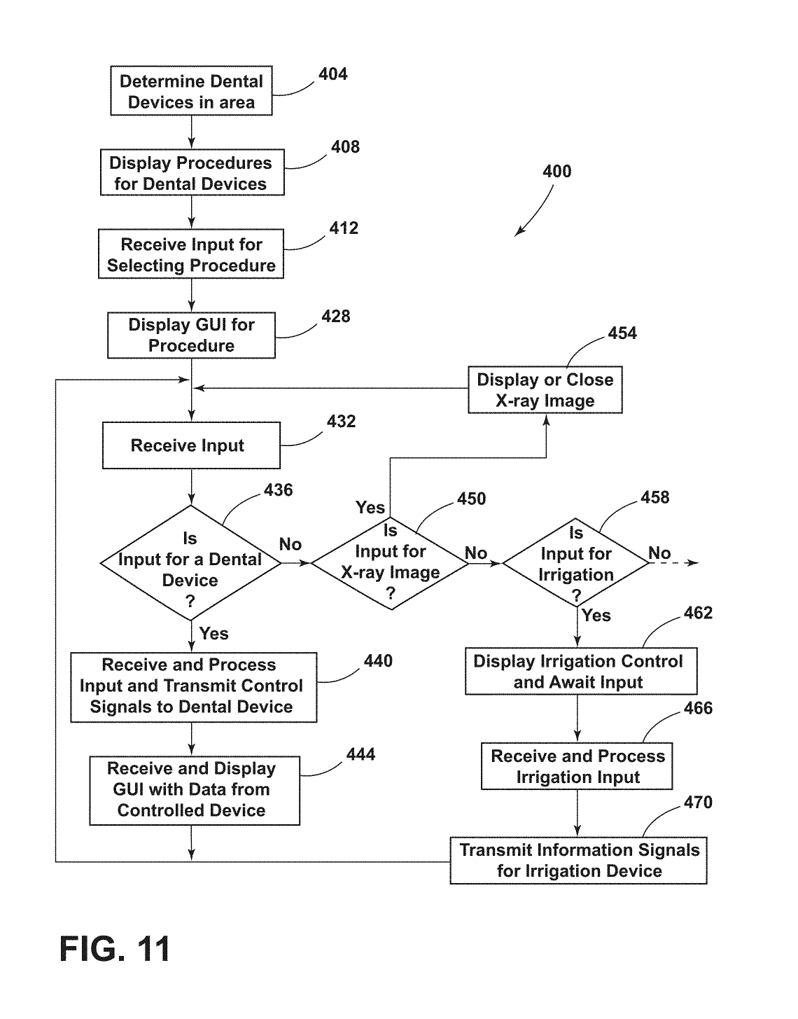

FIG. 11 shows a flow chart 400 for an embodiment of the universal controller 100 that includes selection from multiple procedures. Upon selection, or even before selection upon start-up, the electronic processor 110 of the universal controller 100 executes the method to determine the dental devices disposed in a treatment area nearby using the short range transceiver 120 and to find a connection to a network access point 200 with a local transceiver 124 (step 404). The short range transceiver 120 determines the presence of various devices nearby using Bluetooth or similar communications. The various dental devices were discussed above. In some embodiments, the dental devices are capable of communication with the universal controller 100 via the network access point 200. Further, the network access point 200 simultaneously provides for communication between the local transceiver 124 of the universal controller 100 and the cloud server 230 (step 404).

Thereafter, the universal controller 100 shows a GUI providing a group of procedures on the display 104 that are capable of being performed by detected dental devices (step 408 illustrated in FIG. 11). The user selects a dental procedure on the display 104 to provide an input (step 412) to the universal controller 100. In one embodiment, the procedures to be selected include Cavity, Endodontics, Crown & Bridge, Direct Restoration, Implant, and Apicoectomy. Additional procedures are contemplated.

When the particular procedure is selected and identified by the electronic processor 110, control software for the specific selected procedure and devices utilized in the procedure is provided or linked to the electronic processor 110 from the memory 130, and a graphical user interface for the procedure is provided on the display 104 of the universal controller 100 (step 428 illustrated in FIG. 11). The memory 130 stores information and programs for multiple procedures listed above.

Thereafter, the electronic processor 110 of the universal controller 100 waits to receive an input from the display 104 (step 432 of FIG. 11) for adjusting the control of one of one or more dental devices or to select another device or an image for display.

The electronic processor 110 of the universal controller 100 determines whether the input is for a dental device (decision step 436). If the determination is Yes, the electronic processor 110 processes the input with the specific software of the given dental device and transmits a control signal to the dental device via one of the short range transceiver 120, the local transceiver 124 and the port 128 (step 440). Thereafter, the electronic processor 110 receives information or feedback from the given dental device and displays the information on the GUI reflecting changes in the operation of the device (step 444). Thereafter, the methods returns to await another user input (step 432).

In the instance that the input received from the display 104 is not an input for a dental device (decision step 436), the electronic processor 110 advances to determine whether the input is for a different feature, such as display of a stored X-ray image (decision step 450). If the input is for display of an X-ray image, the method advances to either display the X-ray image for selected tooth/teeth or subsequently to remove the display of the X-ray image from the display 104 of the universal controller 100 (step 454). Thereafter, the electronic processor 110 awaits another input by returning to step 432.

In the instance, the input is not for the display of an X-ray image (decision step 450), the electronic processor 110 determines whether the input is for irrigation (step 458). If not an irrigation selection, the electronic processor 110 proceeds to look for another device/result represented by broken line and an arrow. When the input to the electronic processor 110 is for irrigation, the electronic processor 110 operates to display an irrigation display window on the display 104 of the universal controller 100 (step 462). The electronic processor 110 waits to receive and process an irrigation input (step 466). Thereafter, the electronic processor 110 transmits information signals for the irrigation device (step 470). In one embodiment, the method returns to step 432 to await another input from the GUI on the display 104 of the universal controller 100. Information of the operation of the irrigation device can be recorded and displayed on the GUI on the display 104 in another embodiment.

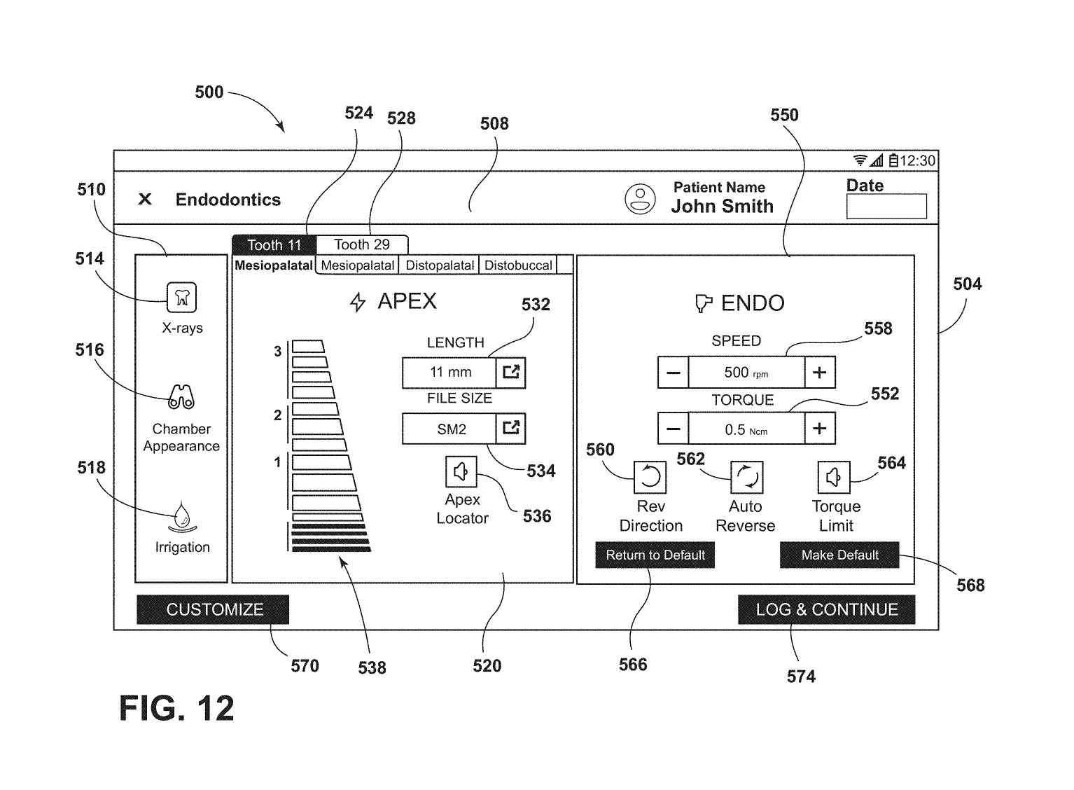

FIG. 12 shows a graphical user interface 500 for the display 104 of the universal controller 100 that generally corresponds with the operation described in FIG. 11. The GUI 500 includes a browser window 504 and a mode bar 508 that displays the operating mode, in this instance “endodontics,” the patient name, date, and time. Thus, FIG. 12 is directed to an endodontics mode of operation. The GUI 500 includes a left section 510 with a vertical oriented group consisting of special virtual selection buttons including X-ray image icon 514, Chamber Appearance icon 516 and Irrigation Icon 518.

A central section of the GUI 500 is directed to a device window 520 for controlling an electronic apex locator and recording patient data for the apex of a specific tooth. The device window 520 includes a pair of tooth tabs 524, 528 for selecting a tooth to be sensed. Further, root canals are listed for a tooth, namely a mesiopalatal cusp for tooth 11 and mesiopalatal, distopalatal and distobuccal cusps for tooth 11. The device window 520 includes a dropdown menu box 532 for generating a list of lengths and for displaying the selected apex length, such as 11 mm. Further, the device window 520 includes a dropdown menu box 534 for generating a list of file sizes and for displaying the selected file size. Further, an apex locator icon 536 is provided in the device window 520 for selectively providing an on/off for the audible warning signal for when the apex has been reached. A bar graph 538 displays results for apex locator operation. In one embodiment, the file sizes and information for each cusp is stored in a memory of at least one from the group consisting of the GUI 500, the logging equipment 220, and the cloud server 230.

A right section of the GUI 500 is directed to a device window 550 for control of maximum operating torque and operating speed of the motor for a tool secured to a handpiece of a delivery unit 30, 70 or of a handpiece system 190. The device window 550 includes a torque display and selection box 552 having a decrement box (−) and an increment box (+) to obtain inputs for adjusting the torque of the motor. Further, the device window 550 includes a speed display and selection box 558 that displays the speed (rpm) of the motor and includes a decrement box (−) and an increment box (+) for touching to change the speed of the motor. Further, the device window 550 includes a reverse direction button or icon 560 for reversing the direction of the motor and an auto reverse button or icon 562. The device window 550 includes a torque limit button or icon 564 for selecting operation of the audible warning when the torque limit is reached. Finally, the device window 550 includes a “Return to Default” virtual button 566 to return to a default setting and a “Make Default” virtual button 568 for setting a new default speed or torque. Thus, a user is capable of selectively controlling two different dental devices of an endodontic procedure directly, as well as directly controlling the apex locator or the endo motor in a one-touch fashion.

Further, the browser window 504 includes a “customize” virtual button 570 for customizing the GUI 500 and operation thereof. Finally, the browser window 504 includes a “Log and Continue” virtual button for logging data and continuing the operation of the endodontics procedure. The settings and operating data for the specific selected devices and instruments that are selected and operated is stored in a patient history file for the specific patient in one embodiment. The information may include lengths and file sizes for specific cusps or roots of a specific tooth and other patient data, including X-rays, medical records, and notes or observations.

Accordingly, the GUI 500 shown in FIG. 12 provides for operating an apex locator and for control of operating torque and operating speed of the motor for a dental device having a tool secured to an instrument, such as a handpiece, in a one touch fashion. In some embodiments, the virtual buttons or selection icons that are displayed in the margin of the GUI 500 of the universal controller include at least one from the group consisting of: X-ray image icon 514, chamber appearance icon 516, and irrigation icon 518.

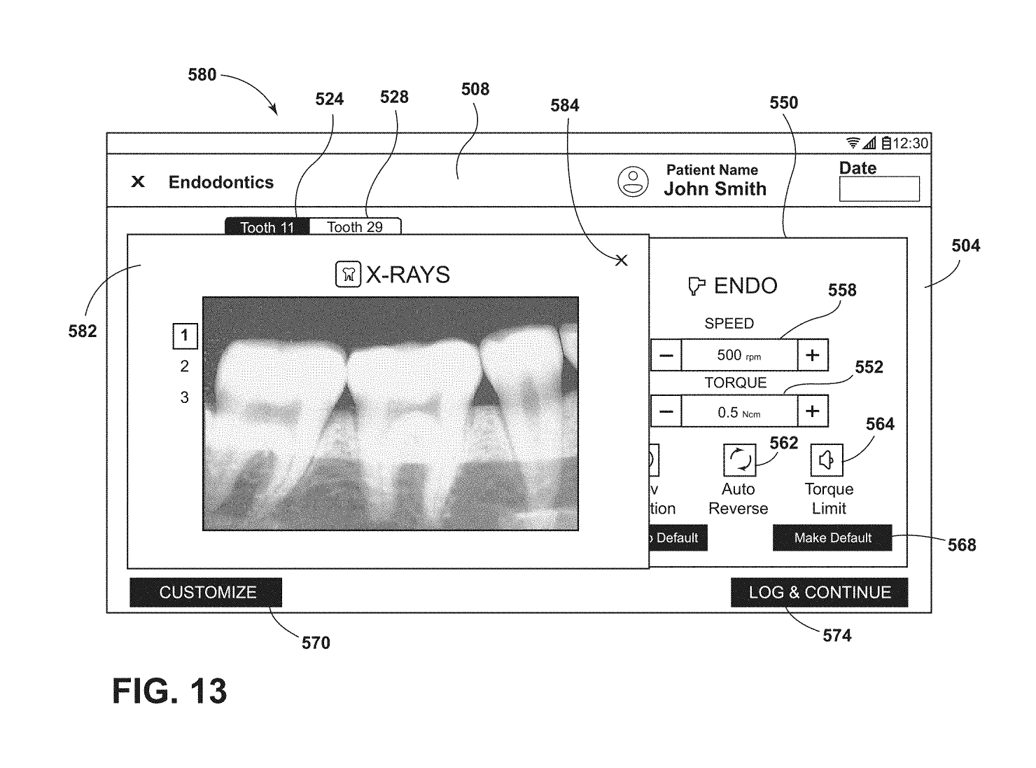

The GUI 580 illustrated in FIG. 13 is provided on the display 104 of the universal controller 100 when the X-ray virtual button 514 illustrated in FIG. 12 is actuated. The X-ray photo image with an X-ray window 582 illustrated in FIG. 13 displays the patient X-ray image of the tooth listed in FIG. 12. The X-ray window 582 as shown in FIG. 13 also effectively overlays a portion of the GUI 500 shown in FIG. 12. A window close “x” virtual button 584 located in the top right of the X-ray window 582 enables closing of the X-ray window and a return to the GUI 500 illustrated in FIG. 12 for the universal controller 100.

In one embodiment, the universal controller 100 obtains the X-ray image for display on GUI 580 through the network access point 200 and from the cloud server 230 that includes a records system that stores patient information. Thus, the universal controller 100 is configured to selectively receive and display patient information, such as patient X-ray images, from the cloud server 230 or other device.

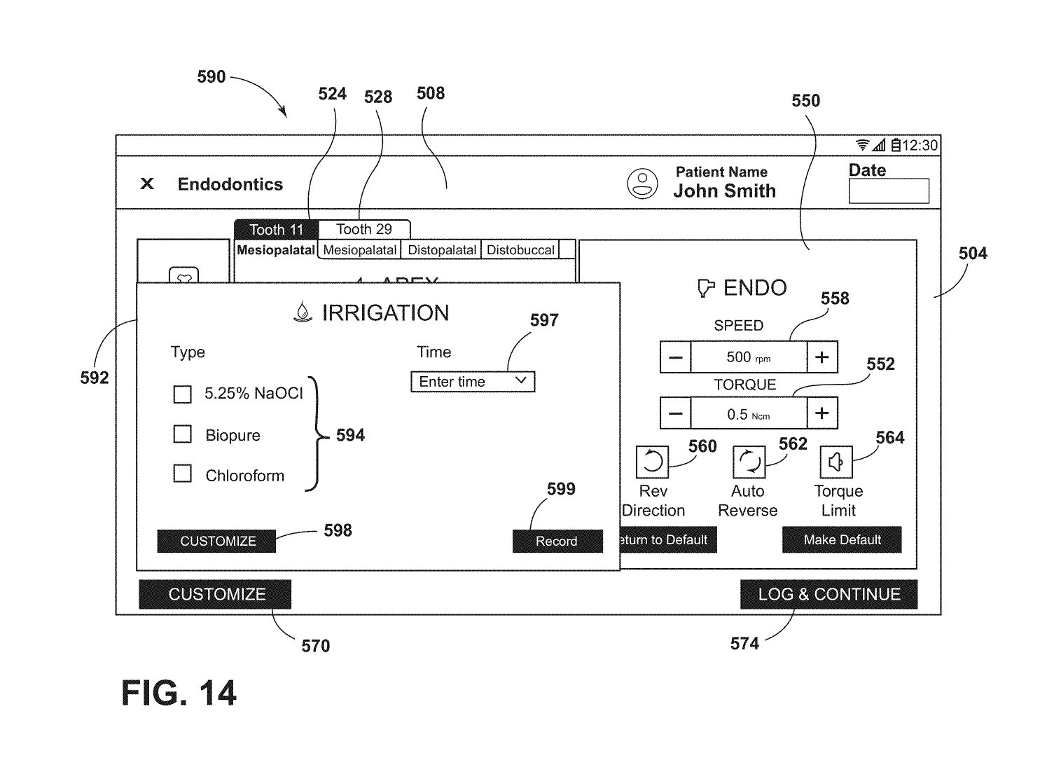

When the irrigation icon 518 is selected in the lower left column illustrated in FIG. 12, an irrigation display window 592 that overlays a portion of the GUI 500 in FIG. 12 is displayed as the GUI 590 illustrated in FIG. 14. The irrigation display window 592 includes a three choice check box 594, wherein only one box can be checked to record the type of irrigation solution. Checking a second box removes the check from a previously checked box. Further, the irrigation display window 592 includes an irrigation time dropdown box 597 for providing a group of irrigation times and for selecting a desired irrigation time. Further, the irrigation display window 592 includes a “customize” virtual button 598 for customizing irrigation and a virtual record button 599 for recording irrigation data. Thus, the irrigation display window 592 enables a selection as to the type of irrigation fluid and a time selection for a user of the universal controller 100 then documenting and storing a record of the irrigation completed during the procedure.

The apex locator that is in communication with the universal controller 100 is operated to measure the depth of the canal during root canal procedures. The depth is also stored during the procedure.

As is known, root canal therapy begins by removal of the organic substrate from the canal. This includes removal of the coronal pulp tissue and radicular pulp tissue. The coronal pulp tissue is removed and straight-line access to the radicular pulp tissue is identified. The radicular pulp tissue is removed with endodontic files and irrigation. Then, infection is prevented by a three-dimensional obturation of the canal to seal the canal system coronally and apically.

An endodontic obturation system provides the options of using heat, vibration or a combination of heat and vibration during obturation. Heat and/or vibration results in a dense, compact filling of the root canal space. In one embodiment, the temperature range for the obturation system illustrated in FIG. 10 is from about 50° C. to 350° C.

Additional Embodiments

The cloud server 230 illustrated in FIG. 7 includes a cloud computer and other processing arrangements. In one embodiment, the cloud server 230 is a logical server that is built, hosted, and delivered through a cloud computing platform over the Internet. The cloud server 230 possesses and exhibits similar capabilities and functionality to a typical server but is accessed remotely from a cloud service provider. In another embodiment, the cloud server 230 is a server located at a dental treatment office for communication with a plurality of treatment units and dental devices.

The dental devices disclosed herein include a group consisting of: dental handpieces provided as stand-alone systems or part of a delivery unit, X-ray sensors, 2-D imaging devices, 3-D panoramic imaging devices, powered treatment chairs 54, lights 50, wireless instruments, endodontic obturation systems, electronic apex locators, maintenance devices, sterilization equipment, irrigation devices, and additional dental equipment provided with a treatment unit 20 in an area within a dental treatment room or group of rooms. In one embodiment, the memory 130 of the universal controller 100 is configured to store control software for at least three from a group of dental devices consisting of: a powered treatment chair 54, lights 50, X-ray sensors, 2-D imaging devices, 3-D imaging devices, wireless instruments, delivery units, apex locators, irrigation devices, obturation systems, maintenance device and sterilization equipment.

The treatment unit 20 shown in FIG. 1 includes two delivery units 30, 70 that support multiple instruments, including handpieces. Additional handpieces, such as provided with the stand-alone dental handpiece system 190, also are provided for use with the treatment unit in some instances.

In one embodiment, the portable universal controller 100 and one or more dental devices define a system for controlling the plurality of dental devices.

FIG. 8 is directed to operating the portable universal controller 100 by selection of dental devices and FIG. 11 is directed to a selection of procedures on a graphical user interface provided on the display 104. Another embodiment enables a user to select among various dental devices and dental procedures from a single graphical user interface provided on the display 104 of the universal controller 100. Thus, the arrangements shown in FIGS. 8 and 11 can be provided as a combination in one embodiment to perform a method for controlling dental devices and performing dental procedures.

In one embodiment, the portable universal controller 100 is a portable tablet. In another embodiment, the portable universal controller 100 includes applications on a cellular phone, a laptop computer or other mobile communication device. While a touchscreen is contemplated for the universal controller 100, in some embodiments an arrangement for moving a cursor on the display 104 with a track pad, mouse, or other implement for selecting inputs is contemplated.

In another embodiment, the short range transceiver 120 and the local transceiver 124 illustrated in FIG. 2 share an antenna for operation as, for instance, a Bluetooth arrangement and a WI-FI arrangement, respectively.

The following examples illustrate example systems, methods and arrangements described herein. Example 1: a universal controller for controlling dental devices, the universal controller comprising: a memory that includes a graphical user interface generator and control software for a plurality of dental devices; a transceiver; a display for displaying graphical user interfaces and for receiving inputs; and an electronic processor connected to the memory, the transceiver, and the display, wherein the electronic processor is configured to provide a graphical user interface to the display in response to a selection received from the display.

Example 2: the universal controller according to example 1, wherein the display comprises a touchscreen.

Example 3: the universal controller according to any of examples 1 and 2, wherein the universal controller is a portable tablet that includes a port for receiving a communication connector, and wherein the electronic processor is configured to operate the transceiver to wirelessly communicate with a selected one of the dental devices.

Example 4: the universal controller according to any of examples 1-3, wherein the electronic processor is configured to synchronize with the dental devices disposed in a dental treatment room, and wherein the graphical user interface provided on the display is for dental devices for a specific selected procedure selected from a group of procedures.

Example 5: the universal controller according to any of examples 1-4, wherein the memory is configured to store control software for at least three from a group of dental devices consisting of: a powered treatment chair, lights, X-ray sensors, 2-D imaging devices, 3-D imaging devices, wireless instruments, delivery units, apex locators, irrigation devices, obturation systems, maintenance devices and sterilization equipment.

Example 6: the universal controller according to any of examples 1-5, wherein the transceiver is a short range transceiver for direct short range wireless communication with each of the dental devices.

Example 7: the universal controller according to any of examples 1-6, including a local transceiver for local communication via a network access point with a cloud server that includes a records system that stores patient information, wherein the universal controller is configured to selectively receive and display patient information and patient images.

Example 8: a system for controlling a plurality of dental devices comprising: a universal controller for controlling a plurality of dental devices, the universal controller comprising: a memory that includes a graphical user interface generator and control software for the plurality of dental devices; a transceiver; a display for displaying graphical user interfaces and for receiving inputs; and an electronic processor connected to the memory, the transceiver, and the display; and a dental device including: an electronic controller for controlling the dental device; a transceiver for wireless communication with the universal controller; and at least one device actuator, wherein the electronic controller is configured to control the device actuator to operate the dental device in response to an input from the universal controller.

Example 9: the system according to example 8, wherein the dental device is free from a touchscreen or a display.

Example 10: the system according to any of examples 8 and 9, the dental device further including a port for receiving a communication connector for wired connection to the universal controller, and wherein the display of the universal controller includes a touchscreen.

Example 11: the system according to any of examples 8-10, wherein the universal controller is a portable tablet.

Example 12: the system according to any of examples 8-11, wherein the dental device is a first dental device and the system further comprises a second dental device including a delivery unit and a handpiece, wherein the universal controller provides the input to operate the first dental device, and wherein the universal controller provides an input to control the handpiece.

Example 13: a method for controlling dental devices with a universal controller, the method comprising: determining dental devices located in an area for communication therewith; displaying a graphical user interface on the universal controller of a plurality of dental devices in the area that are capable of being controlled; in response to selection of one of the dental devices, displaying a graphical user interface on the universal controller that includes control inputs for the selected dental device; and in response to selection of one of the control inputs on the graphical user interface, transmitting a control signal to the selected dental device for operating the selected dental device

Example 14: the method according to example 13, the method including the step of: displaying an operating condition of the selected dental device on the universal controller in response to the universal controller receiving a return signal from the selected dental device.

Example 15: the method according to any of examples 13 and 14, wherein the selected dental device is a delivery unit having a handpiece, and the operating condition displayed on the universal controller includes an operating speed for a tool secured to the handpiece.

Example 16: the method according to any of examples 13-15, including selectively displaying patient data on the display of the universal controller that overlays a portion of the graphical user interface.

Example 17: the method according to any of examples 13-16, wherein the selected dental device is a delivery unit having a handpiece, and the operating condition displayed on the universal controller is an operating torque for a tool secured to the handpiece.

Example 18: the method according to any of examples 13-17, including providing a split screen mode wherein the selected dental device is a first dental device having a first operating condition displayed on the graphical user interface of the universal controller, and wherein a second dental device having a second operating condition is displayed on the graphical user interface of the universal controller, for selective control of either of the first and the second dental devices.

Example 19: the method according to any of examples 13-18, including displaying selection buttons in a margin of the graphical user interface of the universal controller, the selection buttons enabling access to patient data.

Example 20: the method according to any of examples 13-19, wherein the patient data includes an X-ray for display on the graphical user interface in response to selection of the selection button for X-rays.

Example 21: the method according to any of examples 13-20, including displaying selection buttons in a margin of the graphical user interface of the universal controller, wherein actuation of one of the selection buttons displays an irrigation display window on the graphical user interface.

Thus, the embodiments provide, among other things, a universal controller and a method of controlling a plurality of dental devices with the universal controller using a short range transceiver and/or a local transceiver, along with providing patient information to the universal controller. Further, the dental devices do not require a display or input arrangement to synchronize to the universal controller. Various features and embodiments are set forth in the following claims.

What is claimed is:

1. A universal controller for controlling dental devices, the universal controller comprising:
a memory that includes a graphical user interface generator and control software for a plurality of dental devices;
a transceiver;
a display for displaying graphical user interfaces and for receiving inputs; and
an electronic processor connected to the memory, the transceiver, and the display,
wherein the electronic processor is configured to provide a graphical user interface to the display in response to a selection received from the display.

2. The universal controller according to claim 1, wherein the display comprises a touchscreen.

3. The universal controller according to claim 1, wherein the universal controller is a portable tablet that includes a port for receiving a communication connector, and wherein the electronic processor is configured to operate the transceiver to wirelessly communicate with a selected one of the dental devices.

4. The universal controller according to claim 1, wherein the electronic processor is configured to synchronize with the dental devices disposed in a dental treatment room, and wherein the graphical user interface provided on the display is for dental devices for a specific selected procedure selected from a group of procedures.

5. The universal controller according to claim 1, wherein the memory is configured to store control software for at least three from a group of dental devices consisting of: a powered treatment chair, lights, X-ray sensors, 2-D imaging devices, 3-D imaging devices, wireless instruments, delivery units, apex locators, irrigation devices, obturation systems, maintenance devices and sterilization equipment.

6. The universal controller according to claim 1, wherein the transceiver is a short range transceiver for direct short range wireless communication with each of the dental devices.

7. The universal controller according to claim 6, including a local transceiver for local communication via a network access point with a cloud server that includes a records system that stores patient information, wherein the universal controller is configured to selectively receive and display patient information and patient images.

8. A system for controlling a plurality of dental devices comprising:
a universal controller for controlling a plurality of dental devices, the universal controller comprising:
a memory that includes a graphical user interface generator and control software for the plurality of dental devices;
a transceiver;
a display for displaying graphical user interfaces and for receiving inputs; and
an electronic processor connected to the memory, the transceiver, and the display; and
a dental device including:
an electronic controller for controlling the dental device;
a transceiver for wireless communication with the universal controller; and
at least one device actuator, wherein the electronic controller is configured to control the device actuator to operate the dental device in response to an input from the universal controller.

9. The system according to claim 8, wherein the dental device is free from a touchscreen or a display.

10. The system according to claim 8, the dental device further including a port for receiving a communication connector for wired connection to the universal controller, and wherein the display of the universal controller includes a touchscreen.

11. The system according to claim 10, wherein the universal controller is a portable tablet.

12. The system according to claim 11, wherein the dental device is a first dental device and the system further comprises a second dental device including a delivery unit and a handpiece, wherein the universal controller provides the input to operate the first dental device, and wherein the universal controller provides an input to control the handpiece.

13. A method for controlling dental devices with a universal controller, the method comprising:
determining dental devices located in an area for communication therewith;
displaying a graphical user interface on the universal controller of a plurality of dental devices in the area that are capable of being controlled;
in response to selection of one of the dental devices, displaying a graphical user interface on the universal controller that includes control inputs for the selected dental device; and
in response to selection of one of the control inputs on the graphical user interface, transmitting a control signal to the selected dental device for operating the selected dental device.

14. The method according to claim 13, the method including the step of:
displaying an operating condition of the selected dental device on the universal controller in response to the universal controller receiving a return signal from the selected dental device.

15. The method according to claim 14, wherein the selected dental device is a delivery unit having a handpiece, and the operating condition displayed on the universal controller includes an operating speed for a tool secured to the handpiece.

16. The method according to claim 15, including selectively displaying patient data on the display of the universal controller that overlays a portion of the graphical user interface.

17. The method according to claim 14, wherein the selected dental device is a delivery unit having a handpiece, and the operating condition displayed on the universal controller is an operating torque for a tool secured to the handpiece.

18. The method according to claim 14, including providing a split screen mode wherein the selected dental device is a first dental device having a first operating condition displayed on the graphical user interface of the universal controller, and wherein a second dental device having a second operating condition is displayed on the graphical user interface of the universal controller, for selective control of either of the first and the second dental devices.

19. The method according to claim 18, including displaying selection buttons in a margin of the graphical user interface of the universal controller, the selection buttons enabling access to patient data.

20. The method according to claim 19, wherein the patient data includes an X-ray for display on the graphical user interface in response to selection of the selection button for X-rays.

21. The method according to claim 18, including displaying selection buttons in a margin of the graphical user interface of the universal controller, wherein actuation of one of the selection buttons displays an irrigation display window on the graphical user interface.
